(19) United States
(12) Patent Application Publication
(10) Pub. No.: US 2009/0082753 A1
(43) Pub. Date: Mar. 26, 2009

(54) NEEDLE FREE INJECTOR WITH DOSE ADJUSTMENT ASSEMBLY

(75) Inventors: Michael H. Dutcher, Olathe, KS (US); Boris Smolyarov, Voronezh (RU); Victor Rogachev, Voronezh (RU)

Correspondence Address: MAYER BROWN LLP, P.O. BOX 2828, CHICAGO, IL, 60690, US

(73) Assignee: Pulse Needlefree Systems, Inc., Lenexa, KS (US)

(21) Appl. No.: 12/237,297

(22) Filed: Sep. 24, 2008

Related U.S. Application Data
(60) Provisional application No. 60/974,706, filed on Sep. 24, 2007.

(51) Int. Cl. A61M 5/30

(52) U.S. Cl. 604/500; 604/68

(57) ABSTRACT

A needle-free injector with a mechanism to adjust the volume of liquid administered is provided. The injector includes a power base, a fluid path having a plunger to force substances from a cavity defined within the fluid path, and a dose adjustment assembly for adjustably positioning the fluid path relative to the power base to adjust the volume of substance administered to the subject.

### CROSS-REFERENCE TO RELATED APPLICATIONS

This application claims priority to U.S. Provisional Application Ser. No. 60/974,706 filed Sep. 24, 2007, the entire contents of which is hereby incorporated by reference herein.

### FIELD

This disclosure relates to a needle free injector with a dose adjustment assembly for adjusting the dose volume of the medication that the needle free injector will administer to a subject.

### BACKGROUND

For many years, vaccination and administration of medicine has been accomplished by using syringes and needles. However, use of syringes and needles increases the risk of disease transmission among injection recipients. In addition, syringes and needles may cause tissue damage at the site of injection, thereby creating lesions and scar tissue. Particularly with the use of needle injection of animals, injection site lesions may result in losses of tens of millions of dollars each year to meat producers from reduced grade and carcass trim. Further, during injection, needle tips may break causing residual needle fragments to remain in the subject. With animal use, this may further result in needle fragments entering into the food system. Disposable needles and syringes also create hazardous medical waste and waste disposal problems. A further drawback to disposable syringes and needles are the high costs when the units are provided for worldwide use. Many subjects, whether human or animal, have a strong aversion to needle injection. Other drawbacks are associated with the traditional methods of delivering medications using needles and syringes.

Alternative methods of delivering medication have been developed. By way of example, one method is to deliver medication using a needle-free injector. A needle-free injector delivers medication by providing a strong, high pressure blast of the medication through a small orifice, which causes a minute jet stream of the medication to exit the orifice at a velocity and pressure sufficient to allow the transdermal, intradermal, subcutaneous and intramuscular delivery of a medicament. A substantial amount of pressure is needed to create a jet of sufficient velocity to deliver the injectate. However, one drawback of existing needle-free injectors is that they do not allow a user of such systems to easily adjust the dose volume of medication to be injected to the subject. There is therefore presently a need for an easy-to-use, needle-free injector having a dose adjustment assembly for adjusting the dose volume of medication that will be injected to a subject.

### SUMMARY

Utilizing the methods and systems described herein, an injector is provided having a power base, a fluid path including a plunger to force injection substances such as medications from a cavity defined within the fluid path, a contact triggering mechanism and a dose adjustment assembly for adjustably positioning the fluid path relative to the power base to adjust the volume of the substance administered to the subject.

Other embodiments, objects, features and advantages will be set forth in the detailed description of the embodiments that follows, and in part will be apparent from the description, or may be learned by practice, of the claimed invention. These objects and advantages will be realized and attained by the processes and compositions particularly pointed out in the written description and claims hereof. The foregoing Summary has been made with the understanding that it is to be considered as a brief and general synopsis of some of the embodiments disclosed herein, is provided solely for the benefit and convenience of the reader, and is not intended to limit in any manner the scope, or range of equivalents, to which the appended claims are lawfully entitled.

### BRIEF DESCRIPTION OF THE DRAWINGS

The present invention is described in detail below with reference to the attached drawing figures, wherein:

FIG. 1 is a perspective view of a needle free injector;

FIG. 2 is a cross-sectional view of the driving and locking means of the power unit;

FIG. 3 is a perspective view of the fluid path housing and dose adjustment assembly;

FIG. 4 is a cross-sectional view of the fluid path and dose adjustment assembly of FIG. 3;

FIG. 5 is a perspective view of the front flange and the fluid path;

FIG. 6 is a cross-sectional view of the front flange and the fluid path of FIG. 5.

FIG. 7 is a perspective view of the front flange and the fluid path;

FIG. 8 is a cross-sectional view of the front flange and the fluid path of FIG. 7.

### DETAILED DESCRIPTION

While the present invention is capable of being embodied in various forms, the description below of several embodiments is made with the understanding that the present disclosure is to be considered as an exemplification of the claimed subject matter, and is not intended to limit the appended claims to the specific embodiments descrbed herein. The headings used throughout this disclosure are provided for convenience only and are not to be construed to limit the claims in any way. The various embodiments disclosed herein may be combined with other embodiments for the creation and description of yet additional embodiments.

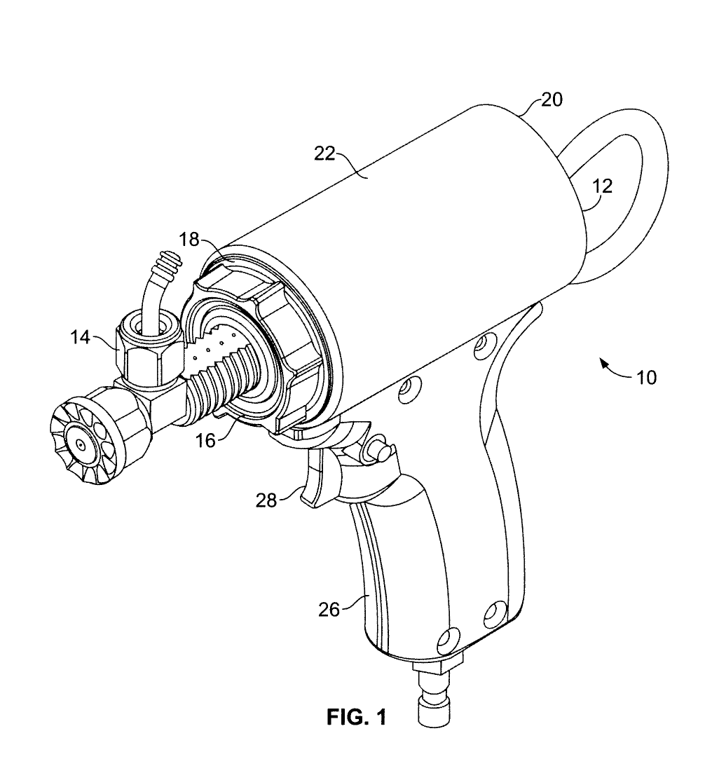

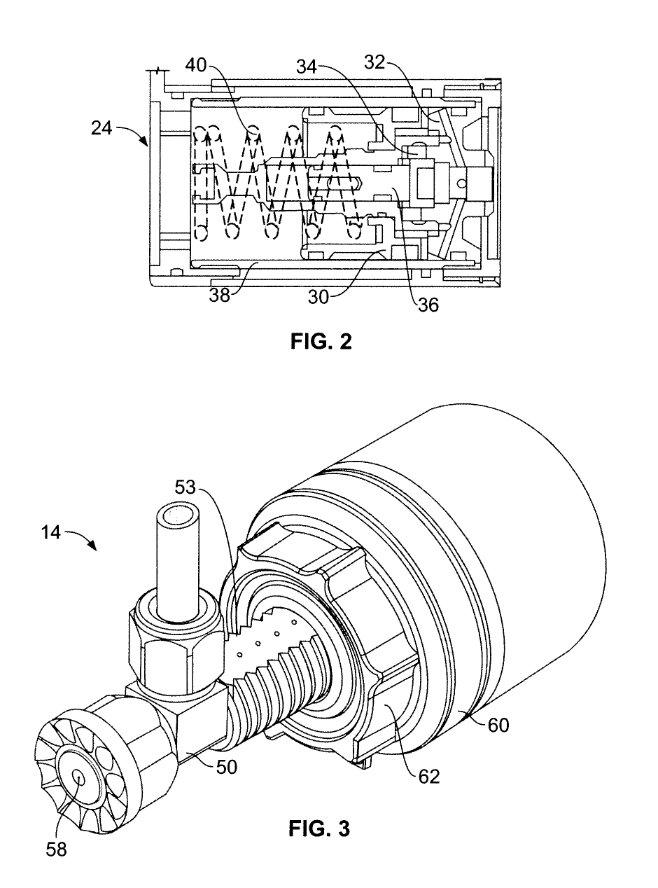

FIG. 1 depicts a needle free injector 10 for injecting a substance into a subject. The injector 10 includes a power unit 12 coupled to an elongated fluid path 14 by a dose adjustment assembly 16. The power unit 12 includes a front flange 18 and a rear flange 20 on opposites ends of a housing 22 within which a driving and locking means 24 (of FIG. 2) is located. The power unit 12 also includes a handle 26 having a safety lever 28 which when enabled permits the power unit 12 to move axially in relation to the handle 26 to trigger the injection of a substance. FIG. 2 depicts the driving and locking means 24 of the exemplary power unit 12. As described in U.S. Pat. No. 6,770,054 entitled “Injector Assembly with Driving Means and Locking Means” issued on Aug. 3, 2004, herein incorporated by referenced in its entirety, an injector is described having a driving means held by a locking means until a predetermined amount of pressure builds in a cavity near the driving means. With reference to FIG. 2 and generally describing the components, a power piston 30 is retained in a retracted first position by a retaining mechanism 32 and a ball lock mechanism 34 until released by a release mechanism 36. Once released, the power piston 30 moves axially within a power cylinder 38 from the retracted first position to an extended second position. A return spring 40 is utilized in re-positioning of the power piston 30 from the extended second position to the retracted first position.

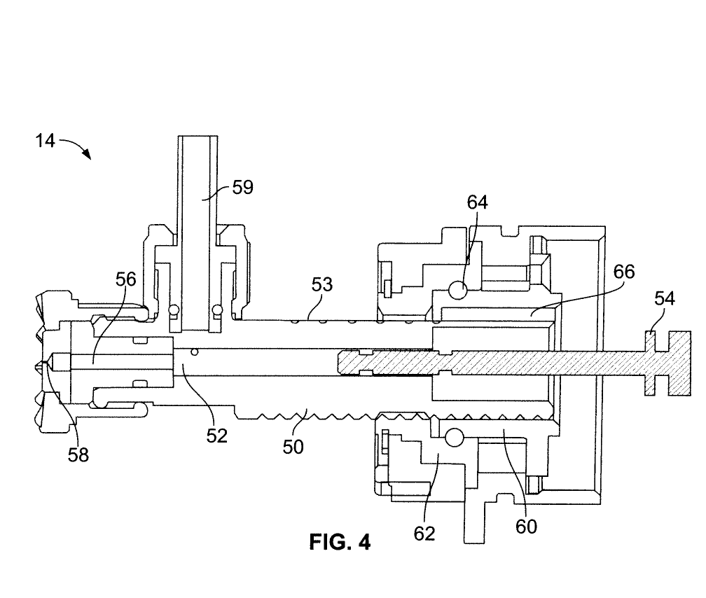

With reference to FIGS. 3 and 4, the fluid path 14 includes a fluid path housing 50 defining a medication chamber 52, and an exterior periphery 53. A medicine piston 54, which is coupled with the power piston 30 of the power unit 12 (FIG. 2), defines a first end of the medication chamber 52 and moves within the fluid path housing 50 to force substances to be injected, such as medications, held in the medication chamber 52 through an outlet check valve 56, out a nozzle orifice 58 and into the subject. The volumetric size of the medication chamber, and thus the volumetric size of the dose of substance, such as a medication, that is to be injected or administered into a subject, is determined by the position of the inserted end of the piston 54 within the fluid path housing 50. In one embodiment, the medicine piston 54 is connected to the power piston 30. When the medicine piston 54 is withdrawn from inside the fluid path housing 50, due to the coupled power piston 30 moving from the extended second position to the retracted first position, the substance to be injected into the subject is drawn into the medication chamber 52 through an inlet check valve 59. However, as described herein, as opposed to the injector disclosed in U.S. Pat. No. 6,770,054, the fluid path housing 50 is modular and axially moveable relative to the power unit 12 as described below. In the preferred embodiment of the present disclosure, the exterior periphery 53 of the fluid path housing 50 contains external threads and may additionally contain markings designating the volume of the medication chamber 52 based upon the position of the fluid path housing 50 relative to the power unit 12 and the inserted end of the medication piston 54 as described below.

Referring to FIG. 4, in an embodiment of the present invention, the dose adjustment assembly 16 adjustably locates the fluid path 14 relative to the power unit 12. In one embodiment, the dose adjustment assembly 16 includes a dose adjustment base 60, a dose adjustment nut 62 and a bearing 64 disposed between the adjustment nut 62 and the bearing 64. In other embodiments, the base 60 is frictionally fit to, or integral with, the front flange 18 of the power unit 12. The base 60 defines a base opening 66 within which the fluid path housing 50 of the fluid path 14 is received. In another embodiment the dose adjustment base 60 further includes a groove (not shown) corresponding to a tongue (not shown) in the fluid path housing 50 of the fluid path 14 to prevent rotational movement of the fluid path 14 within the dose adjustment base 60. In addition to the markings on the exterior periphery 53 of the housing 50 of fluid path 14, the dose adjustment nut 62 may also include markings indicative of the volume of substance to be injected into a subject. The bearing 64 maintains the axial positioning of the dose adjustment nut 62 relative to the base 60 and allows rotation of the nut 62 relative to the base 60. The dose adjustment nut 62 contains internal threads that are complementary to the external threads contained on the exterior periphery 53 of the fluid path housing 50.

In operation, when the dose adjustment nut 62 is rotated about the dose adjustment base 60, the engagement of the threads of nut 62 and the threads of fluid path housing 50 cause the fluid path 14 to move axially relative to the dose adjustment base 60 and power unit 12. When the fluid path 14 is located further within the power unit 12, the volume of the medication chamber 52 is decreased since the inserted end of the medication piston 54, which defines the first end of medication chamber 52, is located closer relative to the end of the chamber 52 associated with the injection nozzle orifice 58. Likewise, when the fluid path 14 is located further away from the power unit 12, the volume of the medication chamber 52 is increased since the end of the medication piston 54 is located farther relative to the end of the chamber 52 associated with the orifice 58. The markings on either the fluid path 14 or the adjustment nut 62 instruct the user of the injector to rotate the nut until the fluid path is positioned correctly thus achieving the desired injection dose volume. Also, the adjustment of the volume in accordance with the present invention does not impact the pressure of the return spring 40 of the power unit or the other operating characteristics of the injector.

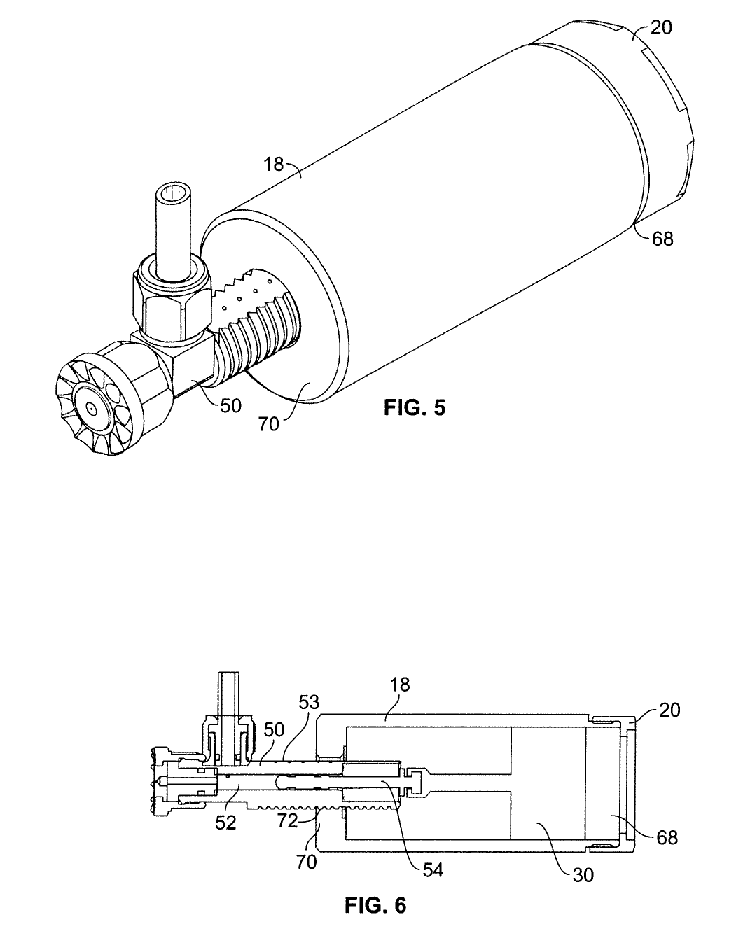

In a further embodiment, FIGS. 5 and 6 illustrate the front flange 18 taking the place of the dose adjustment nut and dose adjustment base while serving a similar function. In this embodiment, the front flange 18 of the needle free injector 10 is substantially a cylindrical cup having an open rear end 68 that engages and seals with rear flange 20, and a partially closed front end 70 that includes an internally threaded dose adjustment orifice 72 disposed therein. Front flange 18 is prevented from undergoing rotational movement relative to the power unit 10. Additionally, in this alternate embodiment, there is no tongue and groove as may be present in other embodiments to prevent the fluid path 14 from undergoing rotational movement. The externally threaded fluid path housing 50 of fluid path 14 engages the complementary internally threaded dose adjustment orifice 72. Engagement of the threads of front flange 18 and the threads of fluid path housing 50 cause the fluid path 14 to be able to move axially relative to the power unit 12 and the inserted end of the medication piston 54 when the fluid path 14 is rotated within the front flange 18. Each incremental rotation of the fluid path 14 results in a corresponding incremental axial movement of fluid path 14 relative to the power unit 12 and the inserted end of the medication piston 54. Accordingly, dose volume adjustment is achieved in this first alternate embodiment by rotating the threaded fluid path 14 within the corresponding threaded front flange 18. In this embodiment, the front flange 18 and the exterior periphery 53 of housing 50 may additionally contain markings designating the volume of the medication chamber 52 based upon the position of the fluid path housing relative to the power unit 12 and the inserted end of the medication piston 54.

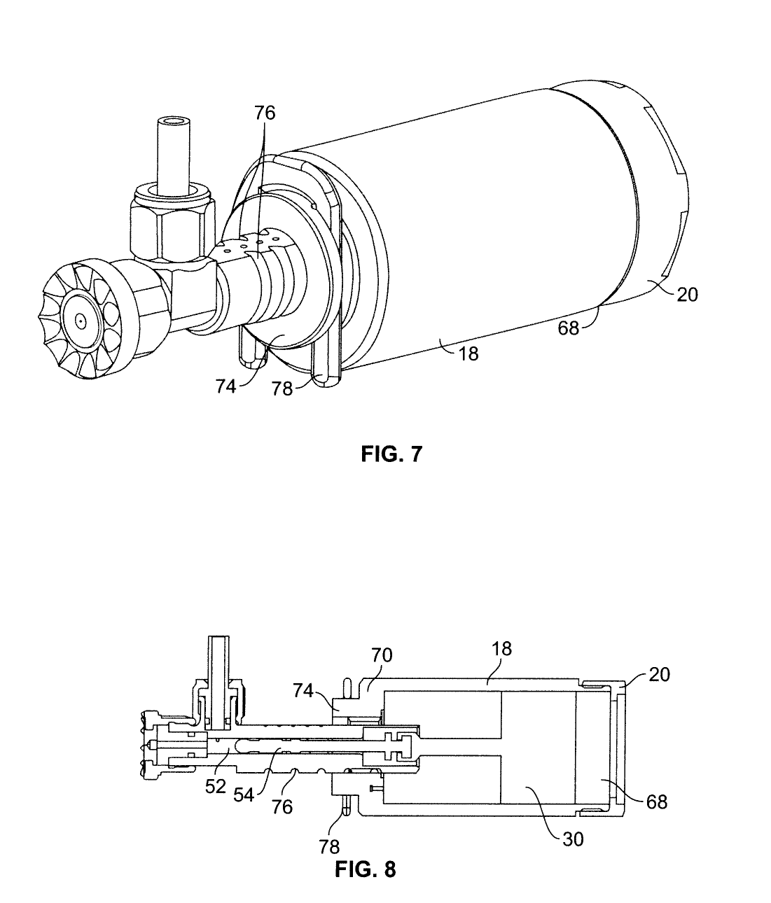

In a further embodiment, FIGS. 7 and 8 illustrate the front flange 18 taking the place of the dose adjustment nut and dose adjustment base while serving a similar function. In this embodiment, the front flange 18 of the needle free injector 10 is substantially a cylindrical cup having an open rear end 68 that engages and seals with rear flange 20, and a partially closed front end 70 that includes a dose adjustment orifice 72 and slotted fluid path retention collar 74. The retention collar 74 may optionally include a groove (not shown) corresponding to a tongue (not shown) in the housing 50 of the fluid path 14 to prevent rotational movement of the fluid path 14 relative to the front flange 18. In this embodiment, the exterior periphery 53 of fluid path housing 50 contains fluid path retention slots 76 disposed therein. In addition, the exterior periphery 53 may additionally contain markings designating the volume of the medication chamber 52 based upon the position of the fluid path housing relative to the power unit 12 and the inserted end of the medication piston 54. In this embodiment, the fluid path retention collar 74 is configured to allow the fluid path 14 to be able to slidably move in an axial direction relative to the power unit 12 and the inserted end of the medication piston 54, when pushed inward or pulled outward by a user. Once the desired position of the fluid path 14 relative to the power unit 12 is set, the position is locked in place by sliding a dose adjustment retention clip 78 into the slots of the retention collar 74 and the corresponding fluid path retention slots 76. Accordingly, dose volume adjustment is achieved in this embodiment by manually positioning the fluid path 14 relative to the power unit 12 and clipping it in place.

The present invention has been described in relation to particular embodiments, which are intended in all respects to be illustrative rather than restrictive. Alternative embodiments will become apparent to those of ordinary skill in the art to which the present invention pertains without departing from its scope. For example, the dose adjustment assembly may be fixedly coupled with the fluid path and engage with the power unit to adjust the volume defined by the medication chamber.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of this disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., such as, preferred, preferably) provided herein, is intended merely to further illustrate the content of the disclosure and does not pose a limitation on the scope of the claims. No language in the specification should be construed as indicating any non-claimed element as essential to the practice of any aspect of the present disclosure.

Alternative embodiments of the claimed disclosure are described herein, including the best mode known to the inventors for practicing the claimed invention. Of these, variations of the disclosed embodiments will become apparent to those of ordinary skill in the art upon reading the foregoing disclosure. The inventors expect skilled artisans to employ such variations as appropriate (e.g., altering or combining features or embodiments), and the inventors intend for the invention to be practiced otherwise than as specifically described herein.

Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

What is claimed is:

1. A needle free injector for delivering a substance to a subject, comprising:
an elongated fluid path housing defining a chamber and having an orifice for delivering the substance to a subject;
a medicine piston moveable within the chamber to force the substance from the chamber;
a power unit adjustably receiving at least a portion of the fluid path housing, wherein the power piston is coupled with the medicine piston and moveable between a retracted position and an extended position;
a contact triggering mechanism to enable automatic firing upon contact; and
a dose adjustment mechanism for selectively locating the fluid path housing relative to the power unit to define the volume of the chamber of the fluid path, whereby the substance in the chamber is delivered to the subject when the power piston is moved from the retracted position to the extended position.

2. The needle free injector of claim 1, wherein the dose adjustment mechanism comprises a dose adjustment base fixedly secured to the power unit and a dose adjustment nut rotatable relative to the dose adjustment base, wherein the dose adjustment nut engages the fluid path housing to locate the housing relative to the power unit.

3. The needle free injector of claim 2, wherein the dose adjustment base defines an opening to receive the fluid path housing.

4. The needle free injector of claim 3, wherein the opening defined in the dose adjustment base includes a groove restricting radial movement of the fluid path housing relative to the dose adjustment base.

5. The dose adjustment mechanism of claim 3, wherein the dose adjustment nut and fluid path housing include corresponding threads, wherein rotation of the dose adjustment nut locates the fluid path housing relative to the power unit.

6. The dose adjustment mechanism of claim 5, wherein the rotational movement of the dose adjustment nut causes axial movement of the fluid path housing in relation to the dose adjustment base.

7. The needle free injector of claim 1, wherein the dose adjustment mechanism comprises a front flange that is fixedly secured to the power unit and wherein the fluid path housing rotatably engages the front flange to locate the housing relative to the power unit.

8. The needle free injector of claim 7, wherein the front flange defines a dose adjustment orifice to receive the fluid path housing.

9. The dose adjustment mechanism of claim 8, wherein the front flange and fluid path housing include corresponding threads, wherein rotation of the fluid path housing within the front flange locates the fluid path housing relative to the power unit.

10. The dose adjustment mechanism of claim 9, wherein the rotational movement of the fluid path housing causes axial movement of the fluid path housing in relation to the power unit.

11. The needle free injector of claim 1, wherein the dose adjustment mechanism comprises a front flange that is fixedly secured to the power unit and wherein the fluid path housing slidably engages the front flange to locate the housing relative to the power unit.

12. The needle free injector of claim 11, wherein the front flange comprises a dose adjustment orifice to receive the fluid path housing and a fluid path retention collar for locking the location of the fluid path in place.

13. The needle free injector of claim 11, wherein the retention collar includes a groove restricting radial movement of the fluid path housing relative to the front flange.

14. The needle free injector of claim 12, wherein the fluid path retention collar and the fluid path housing include corresponding retention slots.

15. The needle free injector of claim 14, wherein the dose adjustment mechanism further comprises a dose adjustment retention clip for engaging the corresponding retention slots of both the fluid path retention collar and the fluid path housing to prevent further axial movement of the fluid path housing and lock the position of the fluid path housing in place relative to the power unit.

16. A method of adjusting the dose of substance to be injected comprising:
providing an injector as described in claim 6;
rotating the dose adjustment nut relative to the dose adjustment base, whereby the rotation of the dose adjustment nut results in the axial movement and positioning of the fluid path housing relative to the power unit and adjustment of the size of the chamber.

17. A method of adjusting the dose of substance to be injected comprising:
providing an injector as described in claim 10;
rotating the fluid path housing relative to the front flange, whereby the rotation of the fluid path housing results in the axial movement and positioning of the fluid path housing relative to the power unit and adjustment of the size of the chamber.

18. A method of adjusting the dose of substance to be injected comprising:
providing an injector as described in claim 15;
removing the dose adjustment retention clip from engaging the corresponding retention slots of both the fluid path retention collar and the fluid path housing;
axially sliding the fluid path housing in relation to the front flange, whereby such sliding action results in the axial movement and positioning of the fluid path housing relative to the power unit and adjustment of the size of the chamber;
inserting the dose adjustment retention clip to engage the corresponding retention slots of both the fluid path retention collar and the fluid path housing so as to prevent further axial movement of the fluid path housing relative to the power unit.

19. A method of administering a needle free injection comprising:
providing a needle free injector as described in any of claims 1 through 15;
adjusting the dose to be injected;
administering the dose to a subject.
